(19) United States
(12) Patent Application Publication
(10) Pub. No.: US 2025/0195028 A1
(43) Pub. Date: Jun. 19, 2025

(54) ULTRASONIC DEVICE

(71) Applicant: SEIKO EPSON CORPORATION, TOKYO (JP)

(72) Inventor: Osamu MURAYAMA, Yamagata-mura (JP)

(21) Appl. No.: 18/980,198

(22) Filed: Dec. 13, 2024

(30) Foreign Application Priority Data
Dec. 13, 2023 (JP) 2023-209939

(51) Int. Cl. A61B 8/08

(52) U.S. Cl. CPC A61B 8/0858

(57) ABSTRACT

An ultrasonic device includes an ultrasonic sensor that is configured to transmit ultrasonic waves into a living body and that is configured to receive ultrasonic waves reflected inside the living body and a housing that includes a window through which the ultrasonic waves pass and that is configured to accommodate the ultrasonic waves therein, wherein the housing includes a reference position indicator and a measurement position indicator and when the reference position indicator is overlapped on a specific part on a body surface of the living body, the measurement position indicator indicates a measurement part on the body surface of the living body.

The present application is based on, and claims priority from JP Application Serial Number 2023-209939, filed Dec. 13, 2023, the disclosure of which is hereby incorporated by reference herein in its entirety.

### BACKGROUND

### 1. Technical Field

The present disclosure relates to an ultrasonic device.

### 2. Related Art

In the related art, an ultrasonic device for measuring muscle thickness or fat thickness using ultrasonic waves has been known (see, for example, JP-A-2001-104302). In the measurement device (ultrasonic device) described in JP-A-2001-104302, on one surface used facing the living body are provided an ultrasonic probe for transmitting and receiving the ultrasonic waves and a reference position unit for positioning the ultrasonic probe at a predetermined position of the living body. In this device, the reference position unit is a protrusion section provided on the same surface as the ultrasonic probe and by aligning the protrusion section to the position of a navel of the living body, it is possible to position the ultrasonic probe at a predetermined part of the living body. That is, when the ultrasonic measurement of the same position is repeatedly performed at intervals of time to observe the temporal change of the tissue of the living body, the contact position of the ultrasonic probe can be positioned at the same position every time.

In the ultrasonic device of JP-A-2001-104302, it is possible to install the ultrasonic device at the same position of the living body, but it is necessary to separately provide the reference position unit constituted by a protrusion section on the surface on which the ultrasonic probe is provided. Therefore, the configuration of the ultrasonic device is complicated and miniaturization is also difficult. When it is desired to designate a plurality of parts of the living body as the measurement parts, it is necessary to provide the reference position units corresponding to these measurement parts and when the plurality of reference position units are provided, the configuration becomes more complicated and the device also increases in size.

### SUMMARY

An ultrasonic device of a first aspect of the disclosure includes an ultrasonic sensor that is configured to transmit ultrasonic waves into a living body and that is configured to receive ultrasonic waves reflected inside the living body and a housing that includes a window through which the ultrasonic waves pass and that is configured to accommodate the ultrasonic waves therein, wherein the housing includes a reference position indicator and a measurement position indicator and when the reference position indicator is overlapped on a specific part on a body surface of the living body, the measurement position indicator indicates a measurement part on the body surface of the living body.

### BRIEF DESCRIPTION OF THE DRAWINGS

FIG. 1 is a plan view showing a schematic configuration of the ultrasonic device of the present embodiment.

FIG. 2 is an explanatory diagram showing an example of a case where a measurement part is determined using the ultrasonic device of the present embodiment.

FIG. 3 is an explanatory diagram showing another example of a case where another measurement part is determined using the ultrasonic device of the present embodiment.

### DESCRIPTION OF EMBODIMENTS

Hereinafter, an embodiment of the present disclosure will be described.

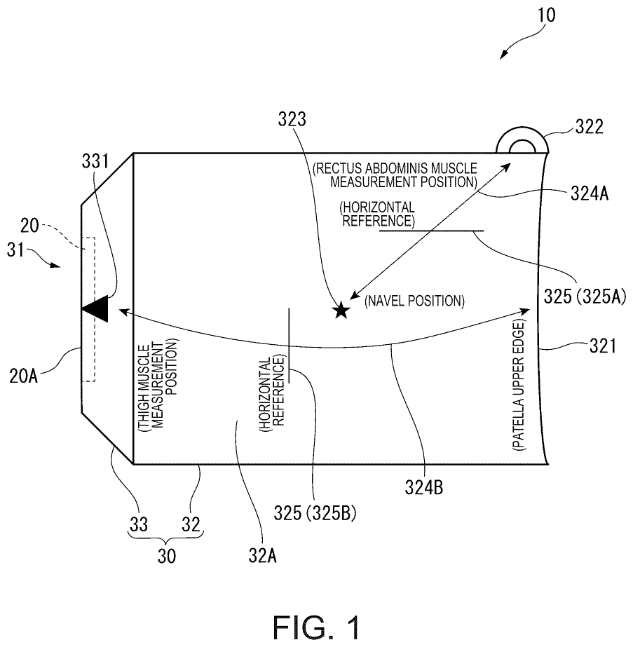

FIG. 1 is a plan view showing a schematic configuration of the ultrasonic device of the present embodiment.

An ultrasonic device 10 includes an ultrasonic sensor 20 and a housing 30.

### Configuration of Ultrasonic Sensor 20

The ultrasonic sensor 20 is a transmission/reception section that transmits ultrasonic waves to a living body and receives ultrasonic waves reflected from within the living body. The ultrasonic sensor 20 is not particularly limited as long as it has a configuration capable of transmitting and receiving the ultrasonic waves to and from, for example, a living body. For example, a bulk-type ultrasonic element may be used in which the ultrasonic waves are transmitted by vibrating a piezoelectric body itself by applying a voltage to the piezoelectric body, and reflected waves are detected by received signal output by distortion of the piezoelectric body itself due to the reflected waves.

Alternatively, it may be a thin film ultrasonic element in which a plurality of ultrasonic transducers, each having a piezoelectric element disposed in a thin film vibrating section, are disposed in an array and the ultrasonic waves are transmitted by vibrating each vibrating section by applying a voltage to the piezoelectric element. In such a thin film ultrasonic element, the received signal is output from the piezoelectric element by the thin film being vibrated by the reflected waves.

When the ultrasonic sensor 20 receives the reflected waves from the living body, the ultrasonic sensor 20 outputs the received signal with a signal value corresponding to the sound pressure of the received ultrasonic waves. Here, in the present embodiment, the ultrasonic sensor 20 continues to receive the reflected waves for a predetermined period from the transmission timing of the ultrasonic waves. Therefore, it becomes a received signal including the change in the signal value according to the time series output from the ultrasonic sensor 20, and the received signal is output to the measurement main section (not shown).

Note that, the measurement main section is a device that is communicably connected to the ultrasonic device 10 in a wired or wireless manner and is configured by, for example, a computer such as a smartphone, a tablet terminal, or a personal computer. In the present embodiment, although detailed description is omitted, the measurement main section receives the received signal from the ultrasonic device 10 and performs various kinds of processing based on the received signal. The various types of processing include, for example, thickness measurement of a predetermined tissue (for example, muscle, fat, etc.) in the living body and recording of changes in the thickness over time, and the like. In addition, generation of an internal tomographic image of the living body based on the received signal from the ultrasonic sensor 20 may be performed.

### Configuration of Housing 30

The housing 30 houses the ultrasonic sensor 20 therein. Specifically, the housing 30 includes a window 31, and a transmission/reception surface 20A of the ultrasonic waves of the ultrasonic sensor 20 is disposed in the window 31. Note that the window 31 may be provided with an acoustic matching layer, an acoustic lens, or the like that also functions to protect the transmission/reception surface 20A. Although not shown, the housing 30 accommodates a drive control circuit that controls the operation of the ultrasonic sensor 20, a communication section that communicates with the measurement main section, a battery that supplies power to each component, and the like.

More specifically, the housing 30, as shown in FIG. 1, includes a main body section 32 having a substantially rectangular shape in plan view and a probe section 33 provided on one end side of the main body section 32.

The main body section 32 houses the drive control circuit, the communication unit, the battery, and the like.

As described above, the main body section 32 is a case of a substantially rectangular shape in plan view as viewed from one direction. However, as shown in FIG. 1, one end surface opposite side from the probe section 33 is a curved surface 321 that is concavely curved in the direction toward the probe section 33. One of the corner portions of the four corners of the housing 30 is provided with, for example, a strap hole 322 that can engage a strap or similar item. Here, in the present embodiment, the curved surface 321 constitutes one of the reference position indicators in the present disclosure, and the strap hole 322 constitutes one of the measurement position indicators. In other words, the curved surface 321 is a part of the housing 30 and also functions as a reference position indicator. The strap hole 322 is a part of the housing 30 and also functions as one of the measurement position indicators.

At a predetermined position on the main body section 32 of the housing 30, a reference position mark 323 is provided. The reference position mark 323 is provided, for example, at the centroid position of one surface (indication surface 32A) of the housing 30 in a plan view in which the housing 30 is viewed from a predetermined direction as shown in FIG. 1. As will be described in detail later, the reference position mark 323 is a mark indicating the reference position when determining a measurement part for performing ultrasonic measurements and is provided by printing, embossing process, or the like. The reference position mark 323 is one of the reference position indicators of the present disclosure.

The probe section 33 is provided on one end side of the main body section 32 and protrudes to the side away from the main body section 32. The protruding tip of the probe section 33 is provided with the window 31 described above. The ultrasonic sensor 20 is housed inside the probe section 33, and the transmission/reception surface 20A of the ultrasonic waves of the ultrasonic sensor 20 is exposed to the outside from the window 31. Note that, as described above, the configuration is not limited to the transmission/reception surface 20A being exposed from the window 31, it is also possible to adopt a configuration such as the transmission/reception surface 20A being protected by the acoustic matching layer or the like.

The probe section 33 is provided with a measurement position mark 331 constituting the measurement position indicator used when performing ultrasonic measurement by printing, embossing process, or the like. The measurement position mark 331 is a mark indicating a part of the outer edge of the housing 30 in a plan view in which the housing 30 is viewed from a predetermined direction as shown in FIG. 1. In other words, it is a mark that indicates a portion of the boundary between the housing 30 and the living body when the housing 30 is in contact with the body surface of the living body and viewed from the housing 30 toward the living body.

Further, in the present embodiment, the housing 30 is provided with a correspondence indicator indicating a combination of the reference position indicator and the measurement position indicator. For example, the strap hole 322, which is the measurement position indicator, is associated with respect the reference position mark 323, which is the reference position indicator, and a correspondence mark 324A is provided as the correspondence indicator indicating this.

The measurement position mark 331, which is a measurement position indicator, is associated with the curved surface 321, which is a reference position indicator, and a correspondence mark 324B is provided as the correspondence indicator indicating this.

These correspondence marks 324A and 324B are provided on the housing 30 by printing, embossing process, or the like.

As the correspondence marks 324A and 324B, a direction instruction section 325 may be further provided that instructs about the inclination direction of the housing 30 for when the housing 30 is brought in contact with the living body so that the reference position indicator (reference position mark 323 and curved surface 321) overlaps a specific part.

For example, in the example shown in FIG. 1, when the living body (human body) is standing, the horizontal instruction lines 325A and 325B indicating the direction of the housing 30 are provided for when the housing 30 is brought in contact with the living body such that the reference position indicator overlaps the specific part.

### Determining Method of Measurement Part for Performing Ultrasonic Measurement

Next, a determining method of a position (measurement part) at which the ultrasonic device 10 is installed with respect to the living body will be described.

By using the ultrasonic device 10 of the present embodiment, it is possible to continuously and repeatedly perform ultrasonic measurement at the predetermined measurement part of the living body and observe a change in tissue within the living body. For example, a case will be described in which a change over time in the thickness of a predetermined tissue (for example, muscle or fat) in the living body is measured (follow-up observations). The user brings the ultrasonic device 10 into contact with the measurement part corresponding to the tissue and performs ultrasonic measurement. In order to perform the follow-up observation, it is necessary to perform ultrasonic measurement by bringing the ultrasonic device 10 into contact with the same measurement part at a predetermined cycle (for example, every other day, every other week, or the like). In this case, follow-up observation on the progress of the target tissue cannot be performed correctly if the ultrasonic device 10 is in contact with a different position for each measurement, so it is necessary to bring the ultrasonic device 10 into contact with the same position for each measurement.

In the present embodiment, in order to specify the measurement part, the measurement part is specified using a part of the housing 30 without using a separate configuration for positioning in the ultrasonic device 10.

Hereinafter, as an example, a determining method of the measurement part in the case of performing a follow-up observation of the rectus abdominis muscle and the thigh muscle will be described.

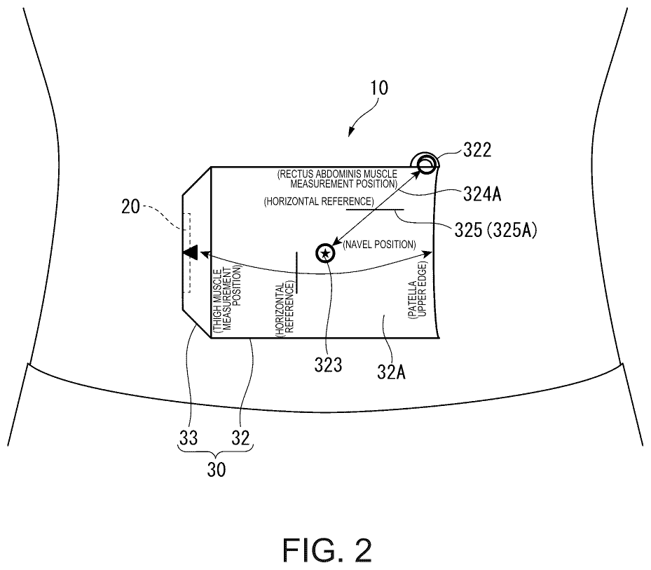

FIG. 2 is a diagram showing an example of a case where the measurement part is determined using the ultrasonic device 10 of the present embodiment and is an explanatory diagram for explaining the measurement part determination method of the rectus abdominis muscle.

When the rectus abdominis muscle is measured using the ultrasonic device 10 of the present embodiment, the measurement part is determined by the reference position mark 323 associated with the correspondence mark 324A and the strap hole 322.

As described above, the reference position mark 323 is provided on the housing 30 by printing, embossing process, or the like, and the strap hole 322 is provided at one of the four corners of the housing 30. Here, the distance between the reference position mark 323 and the strap hole 322 is set to a general distance from the navel to the rectus abdominis muscle. As the general distance, for example, an average value of the distance between the abdominal position immediately above the rectus abdominis muscle and the navel position can be exemplified. The ultrasonic device 10 is categorized for use by adult males, adult females, children, and the like, and the distance between the reference position mark 323 and the strap hole 322 may be determined accordingly for each category.

In the present embodiment, the user sets the indication surface 32A of the housing 30 on which the reference position mark 323 will be indicated as the front side and brings the back surface into contact with the abdomen (see FIG. 2). At this time, the housing 30 is brought into contact such that the reference position mark 323 is positioned at the navel position and the horizontal instruction line 325A is horizontal (when the measurement target person is standing). Then, the user marks the body surface of the living body corresponding to the position of the strap hole 322 and determines the marked position as the measurement position of the rectus abdominis muscle.

Thereafter, the ultrasonic measurement is performed by bringing the ultrasonic device 10 into contact with the marked position. Thereafter, by performing the same procedure when measuring the rectus abdominis muscle, it is possible to perform follow-up observation at the same position of the rectus abdominis muscle.

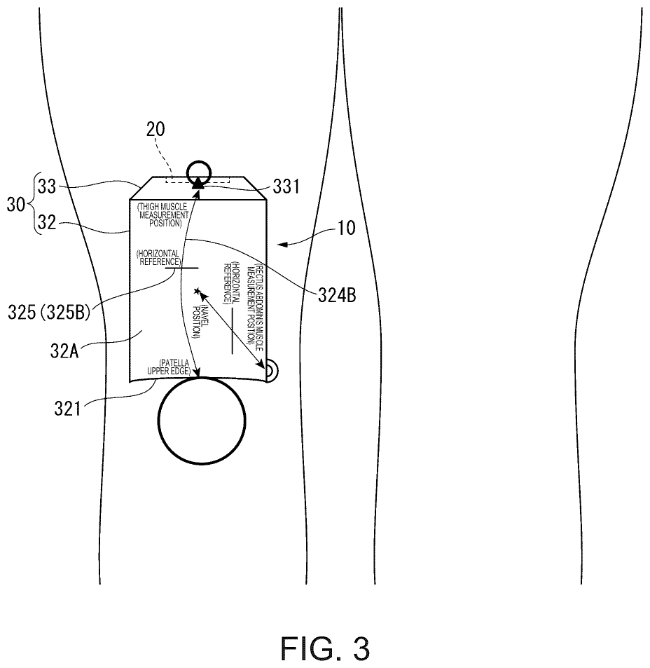

FIG. 3 is a diagram showing another example of determining the measurement part using the ultrasonic device 10 of the present embodiment and is an explanatory diagram for explaining a method of determining the measurement part of a thigh muscle.

When the thigh muscle is measured using the ultrasonic device 10 of the present embodiment, the measurement part is determined by the curved surface 321 associated with the correspondence mark 324B and the measurement position mark 331.

As described above, the curved surface 321 is one end surface of the housing 30, and the measurement position mark 331 is provided on the probe section 33 of the housing 30. Here, the distance from the curved surface 321 to the measurement position mark 331 is set to a general distance from an upper edge of the patella to the thigh muscle. As described above, the ultrasonic device 10 is categorized for use with adult males, adult females, children, and the like, and the distance from the curved surface 321 to the measurement position mark 331 may be determined accordingly for each category.

In the present embodiment, the user sets the indication surface 32A of the housing 30 on which the measurement position mark 331 is indicated as the front surface and brings the back surface into contact with the thighs (see FIG. 3). At this time, the housing 30 is brought into contact such that the curved surface 321 is disposed along the upper edge of the patella and the horizontal instruction line 325B is horizontal (when the measurement target person is standing). Then, the user marks the body surface of the living body corresponding to the position of the measurement position mark 331 and determines the marked position as the measurement position of the thigh muscle.

Thereafter, the ultrasonic measurement is performed by bringing the ultrasonic device 10 into contact with the marked position. Thereafter, by performing the same procedure when measuring the thigh muscle, it is possible to perform follow-up observation on the same position of the thigh muscle.

### Operational Effects of Present Embodiment

The ultrasonic device 10 of the present embodiment includes the ultrasonic sensor 20 and the housing 30 is configured to house the ultrasonic sensor 20. The ultrasonic sensor 20 transmits the ultrasonic waves into the living body and receives the ultrasonic waves reflected within the living body. The housing 30 includes the window 31 through which the ultrasonic waves pass and is provided with the reference position mark 323 and the curved surface 321, which are reference position indicators, and the strap hole 322 and the measurement position mark 331, which are measurement position indicators. The strap hole 322 and the measurement position mark 331, which are measurement position indicators, indicate a measurement part on the body surface of the living body when the reference position mark 323 and the curved surface 321, which are reference position indicators, overlap the specific part on the body surface of the living body.

That is, the distance between the reference position indicator and the measurement position indicator is a general average distance between the specific part of the living body (for example, the navel position, the upper edge of the patella, etc.) and the desired measurement part (for example, a position corresponding to the rectus abdominis muscle or a position corresponding to the thigh muscle). Therefore, by overlapping the reference position indicator on the specific part, the user can easily recognize the position of the measurement position indicator as the measurement part. By marking the position, the user can bring the ultrasonic device 10 into contact with the same position each time the ultrasonic measurement is performed, allowing for desirable observation of tissue within the living body.

In the present embodiment, since a part of the housing 30 is used as the reference position indicator or the measurement position indicator, it is not necessary to separately provide an instruction means for instructing the measurement part. Therefore, it is possible to suppress complication or an increase in size of the configuration of the ultrasonic device 10 and to determine the measurement part with a simple configuration. A measure, a jig, and the like can be made unnecessary.

In the present embodiment, as the reference position indicator, the mark image indicated on the exterior surface of the housing 30 (the reference position mark 323, or the curved surface 321 which is a part of the shape of the housing 30) is used.

The reference position mark 323 is a mark indicated on housing 30 by printing or embossing process and is not incorporated by adding a separate special structure as the reference position indicator. Similarly, the curved surface 321 is a configuration of a part of the housing 30 and is not incorporated by adding a separate special structure as the reference position indicator to the housing 30. Thus, as described above, as compared with the case of providing an instruction means or the like of another configuration, it is possible to simplify the configuration.

In the present embodiment, the measurement position indicator is the measurement position mark 331 indicating a part of the boundary between the housing 30 and the living body when in a state of the housing 30 is in contact with the body surface of the living body and viewed from the housing 30 toward the living body, or the strap hole 322 which is a corner portion of the housing 30.

The measurement position mark 331 is a mark indicated on the housing 30 by printing or embossing process and is not incorporated by adding a separate special structure as the measurement position indicator. Similarly, the strap hole 322 is one of the four corners that is a part of the housing 30 and is not incorporated by adding a separate special structure as the measurement position indicator to the housing 30. Thus, as described above, as compared with the case of providing a positioning means of another configuration, it is possible to simplify the configuration. Since the measurement position indicator is a part of the boundary between the living body and the housing 30 when the housing 30 is brought into contact with the living body, it is easy to perform marking on the body surface of the living body at the portion indicated by the measurement position indicator, and it is possible to suppress the inconvenience of losing sight of the specified measurement part.

Note that, in the present embodiment, although the configuration in which the strap hole 322 is provided in one of the four corners of the housing 30, it may not be provided with the strap hole 322, in this case, a measurement position mark indicating one of the four corners of the housing 30 may be provided.

In the present embodiment, the housing 30 is provided with a plurality of measurement position indicators corresponding to different measurement parts of the living body, and the reference position indicator is provided corresponding to each of the plurality of measurement position indicators. For example, the reference position mark 323 is provided for the strap hole 322 corresponding to the measurement of the rectus abdominis muscle, and the curved surface 321 is provided for the measurement position mark 331 corresponding to the measurement of the thigh muscle.

As a result, in the present embodiment, measurement by the ultrasonic measurement can be performed using the same ultrasonic device 10 in both cases of performing the follow-up observation of the rectus abdominis muscle is desired and the follow-up observation of the thigh muscle is desired. In the present embodiment, the rectus abdominis muscle and the thigh muscle are taken as examples, but in order to set the measurement part corresponding to other tissues, other portions of the housing 30 may be used as the measurement position indicator or the reference position indicator. In any case, the plurality of measurement parts can be easily determined by the single ultrasonic device 10.

In the present embodiment, regarding the determination of the measurement part corresponding to the rectus abdominis muscle, the correspondence mark 324A (correspondence indicator) is further provided which indicates the correspondence relationship between the reference position mark 323, which is the reference position indicator, and the strap hole 322, which is the measurement position indicator corresponding to the reference position mark 323. Similarly, regarding the determination of the measurement part corresponding to the thigh muscle, the correspondence mark 324B (correspondence indicator) is further provided which indicates the correspondence relationship between the curved surface 321, which is the reference position indicator, and the measurement position mark 331, which is the measurement position indicator corresponding to the curved surface 321.

Accordingly, when the ultrasonic device 10 is installed such that the reference position indicator overlaps the specific part of the living body, the user can easily confirm which position of the housing 30 should be determined as the measurement part.

### Modification

It should be noted that the present disclosure is not limited to the embodiments described above, and modifications and improvements within the scope of attaining the object of the present disclosure, and configurations obtained by appropriately combining the embodiments are included in the present disclosure.

### Modification 1

In the above described embodiment, the reference position indicator is exemplified by the reference position mark 323 and the curved surface 321, but other portions may be used as well. For example, regarding the determination of the measurement part corresponding to the rectus abdominis muscle, a part of the boundary between the housing 30 and the living body when the ultrasonic device 10 is brought in contact with the body surface of the living body and the living body is viewed from the ultrasonic device 10 side may be used as the measurement position indicator.

Only one reference position indicator may be provided. For example, it may be such that only the reference position mark 323 is provided and the plurality of measurement position indicators may be provided for the reference position mark 323. For example, the strap hole 322, which is one of the four corners of the housing 30, may be used as the measurement position indicator when the rectus abdominis muscle is measured, and the measurement position mark 331 may be used as the measurement position indicator when the pectoral muscle is measured. In this case, in the measurement of the pectoral muscle, the reference position mark 323 is disposed to overlap the armpit.

### Modification 2

The strap hole 322 and the measurement position mark 331 are exemplified as the measurement position indicator, but other portions may be used.

Even in the case of the ultrasonic device 10 corresponding to the plurality of measurement parts, only one measurement position indicator may be provided and the plurality of reference position indicators may be provided corresponding to the measurement parts.

### Modification 3

In the above described embodiment, an example is shown in which the housing 30 is provided with the horizontal instruction lines 325A and 325B as the direction instruction section 325, and the housing 30 is brought into contact so that the reference position indicator overlaps with the specific part and the horizontal instruction lines 325A and 325B are in the horizontal direction, but this is not a limitation. For example, as the direction instruction section 325, a vertical instruction line indicating a vertical direction may be provided. In this case, the housing 30 brought into contact so that the reference position indicator overlaps on the specific part and the vertical instruction line is in the vertical direction.

### Modification 4

In the above described embodiment, an example in which the measurement main section is configured as a separate body from the ultrasonic device 10 is described. However, a calculation circuit section may be provided in the housing 30 of the ultrasonic device 10, and the calculation circuit section may function as the measurement main section. In this case, the ultrasonic device 10 performs various kinds of processing and outputs the results to a display device or another terminal device.

The display device as an output destination may be separately provided in the housing 30. In this case, a display may be provided on the indication surface 32A of the housing 30 and a measurement position mark 331, a reference position mark 323, correspondence marks 324A and 324B, horizontal instruction lines 325A and 325B, and the like (to be described later) may be displayed on the display. In the present configuration, it may a feature that causes the user to select the measurement part to be measured, and that causes display of only the measurement position indicator, the reference position indicator, and the horizontal instruction line (direction instruction section) corresponding to the selected measurement part.

### SUMMARY OF PRESENT DISCLOSURE

The ultrasonic device according to a first aspect of the disclosure includes an ultrasonic sensor that is configured to transmit ultrasonic waves into a living body and that is configured to receive ultrasonic waves reflected inside the living body and a housing that includes a window through which the ultrasonic waves pass and that is configured to accommodate the ultrasonic waves therein, wherein the housing includes a reference position indicator and a measurement position indicator and when the reference position indicator is overlapped on a specific part on a body surface of the living body, the measurement position indicator indicates a measurement part on the body surface of the living body.

As a result, the user can easily recognize the position of the measurement position indicator as the measurement part by overlapping the reference position indicator on the specific part of the living body without using a dedicated jig or measure. Since a part of the housing is used as the reference position indicator or the measurement position indicator, there is no need to provide separate instruction means for instructing the measurement part, which can prevent the ultrasonic device from becoming more complex and larger in size, allowing the measurement part to be determined with a simple configuration.

In the ultrasound device of the present aspect, it is desirable that the reference position indicator is a mark image indicated on the exterior surface of the housing or is a part of the shape of the housing.

As a result, as compared with the case of providing an instruction means of another configuration for determining the measurement part, it is possible to simplify the configuration.

In the ultrasound device of the present aspect, it is desirable that the measurement position indicator is a part of a boundary between the housing and the living body when in a state of the housing being in contact with the body surface of the living body and viewing from the housing toward the living body, or is a corner of the housing.

As a result, as compared with the case of providing an instruction means of another configuration for determining the measurement part, it is possible to simplify the configuration. Since the measurement position indicator is a part of the boundary between the living body and the housing when the housing 30 is brought into contact with the living body, it is easy to perform marking on the body surface of the living body at the portion indicated by the measurement position indicator, and it is possible to suppress the inconvenience of losing sight of the specified measurement part.

In the ultrasound device of the present aspect, it is desirable that the housing includes a plurality of measurement position indicators corresponding to different measurement parts of the living body.

Thus, the plurality of measurement parts can be determined by one ultrasonic device. Therefore, for example, as compared with the case of using a jig for the rectus abdominis muscle, a jig for the thigh muscle, or the like, the measurement part can be determined more easily.

In the ultrasound device of the present aspect, it is desirable that the housing includes a plurality of reference position indicators corresponding to each of the plurality of measurement position indicators.

Thus, the plurality of measurement parts can be determined by the housing of one ultrasonic device.

In the ultrasound device of the present aspect, it is desirable that the ultrasonic device further includes a correspondence indicator configured to indicate the correspondence between one of the reference position indicators and one of the measurement position indicators that correspond to the one of the reference position indicators.

Thus, when providing the plurality of measurement position indicators in the housing or when providing a reference position indicator corresponding to the plurality of measurement position indicators, the correspondence becomes easily visually recognizable to the user and it is possible to suppress the inconvenience of missetting the measurement part.

What is claimed is:

1. An ultrasonic device comprising:
an ultrasonic sensor that is configured to transmit ultrasonic waves into a living body and that is configured to receive ultrasonic waves reflected inside the living body and
a housing that includes a window through which the ultrasonic waves pass and that is configured to accommodate the ultrasonic waves therein, wherein
the housing includes a reference position indicator and a measurement position indicator and
when the reference position indicator is overlapped on a specific part on a body surface of the living body, the measurement position indicator indicates a measurement part on the body surface of the living body.

2. The ultrasonic device according to claim 1, wherein
the reference position indicator is a mark image indicated on the exterior surface of the housing or is a part of the shape of the housing.

3. The ultrasonic device according to claim 1, wherein
the measurement position indicator is a part of a boundary between the housing and the living body when in a state of the housing being in contact with the body surface of the living body and viewing from the housing toward the living body, or is a corner of the housing.

4. The ultrasonic device according to claim 1, wherein
the housing includes a plurality of measurement position indicators corresponding to different measurement parts of the living body.

5. The ultrasonic device according to claim 4, wherein
the housing includes a plurality of reference position indicators corresponding to each of the plurality of measurement position indicators.

6. The ultrasonic device according to claim 5, further comprising
a correspondence indicator configured to indicate the correspondence between one of the reference position indicators and one of the measurement position indicators that correspond to the one of the reference position indicators.
